(19) United States
(12) Patent Application Publication
(10) Pub. No.: US 2012/0035277 A1
(43) Pub. Date: Feb. 9, 2012

(54) LIQUID-FILLED CHEWABLE SUPPLEMENT

(75) Inventor: Judy Davis, San Clemente, CA (US)

(73) Assignee: Hero Nutritionals, LLC, San Clemente, CA (US)

(21) Appl. No.: 13/198,596

(22) Filed: Aug. 4, 2011

Related U.S. Application Data
(60) Provisional application No. 61/370,743, filed on Aug. 4, 2010.

(51) Int. Cl. A61K 47/00; B05D 3/00; A23L 1/302; B05D 5/00; A23L 1/00; A23L 1/30

(52) U.S. Cl. 514/777; 426/89; 426/72; 426/61; 426/103; 426/289; 427/2.14

(57) ABSTRACT

A liquid-filled chewable composition for the oral delivery of nutritional supplements and/or pharmaceuticals is provided. The chewable composition includes a delivery vehicle and an active ingredient incorporated therein. The delivery vehicle may include an organic or non-organic gummy candy including a binding agent, sweetener, flavoring, and/or coloring. The active ingredient may include a predetermined amount of any combination of pharmaceuticals, nutraceuticals, vitamins, minerals, antioxidants, soluble and insoluble fiber, herbs, plants, probiotics, prebiotics, antioxidants, amino acids, fatty acids, digestive enzymes, dietary supplements, or any other health promoting ingredient. The delivery vehicle may also include a predetermined amount of at least one nutritional supplement and/or pharmaceutical compound.

### RELATED APPLICATIONS

This application claims priority from U.S. Provisional Application No. 61/370,743, filed on Aug. 4, 2010, titled LIQUID-FILLED CHEWABLE SUPPLEMENT, which application is incorporated in its entirety by reference herein.

### BACKGROUND OF THE INVENTION

1. Field of the Invention

The invention relates generally to a chewable dietary supplement, and more particularly to a chewable composition with a liquid-filled center for the oral delivery of dietary supplements and pharmaceutical compounds and a method for manufacturing the same.

2. Related Art

Recently, chewable supplements have been manufactured and sold in the form a gummy candy supplement. Now a selection of vitamins and other dietary supplements are being manufactured and sold in a chewable gummy form, including both children and adult supplements. The introduction of gummy supplements into the marketplace has been particularly helpful in getting children to take daily vitamin supplements. For adults that do not like swallowing pills, gummy supplements have also provided a non-pill alternative for adults to get their daily vitamin requirements.

Although gummy candy was first introduced in 1920 as “gummy bears,” it was not until very recently that gummy candy was first utilized, by Hero Nutritionals, LLC, San Clemente, Calif., as a delivery system for dietary supplements. Traditional gummy candy is made from a gelatin base, which is similar to the base found in soft caramels, marshmallows, foam-filled wafers, licorice, wine gums, pastilles, chocolate coated mallows and a host of other sweets. Gelatin is a protein derived from animal tissue that forms thick solutions or gels when placed in water. When used in gummy candy, gelatin serves as a binding agent that gives the candy its elasticity and desired chewy consistency.

In addition to gelatin, gummy candies are generally made from a blend of water, sweeteners (e.g., corn starch, corn syrup, and/or sugar), flavors, and colors. When mass produced, a gelatin base or stock is first mixed and pumped into a special candy cooker that cooks the gelatin base by steam. Then, the cooker pumps the gelatin base into a vacuum chamber to remove excess moisture. From the vacuum chamber, the cooked candy moves to a mixing station where colors, flavors, acids, and fruit concentrates are mixed into the cooked candy. Next, a starch molding machine, commonly known as a mogul, pumps the candy stock into starch filled mold boards that shape the candies. After curing, the gummies are removed from the molds and then packaged, delivered, and sold.

Gummy vitamins have become a favorite among children as well as adults. Gummy vitamins have grown in popularity because of their unique combination of sweetness and nutritional value. The popularity of gummy vitamins has rivaled those of other confectionary products. One such rival is liquid-filled chewing gum. Liquid-filled gums, such as Chewels®, Tidal Wave®, Freshen Up®, and most recently Trident Splash®, generally include a solid, chewing gum-like outer portion or shell, and a soft or liquid center—typically a flavored liquid have an syrup-like consistency. Liquid-filled gums are popular because they produce an initial strong sweet taste as the liquid center is quickly released into the oral cavity (i.e., initial liquid “burst”) when the gum is consumed. Thus, liquid-filled gums are effective in delivering their active ingredients, i.e., liquid sweetener, into the oral cavity of a consumer.

Even with the growing popularity of gummy supplements, to date, no one has utilized gummy candy with a liquid-filled center as a delivery system for dietary supplements and/or pharmaceuticals. Thus, a need exists in the art for a safe, easily digestible and palatable delivery system that enables the effective oral delivery of dietary supplements and/or pharmaceuticals.

### SUMMARY

An edible, digestible composition is provided that includes a chewable composition including a center-fill composition comprising a flavored liquid ingredient, and a chewable shell surrounding the center-fill composition, the chewable shell comprising a binding agent, flavoring ingredient, and a sweetener, and at least one health promoting ingredient. By ingesting the gummy candy, the consumer is able to directly supply his or her body with active health ingredients

In some implementations, composition may be in the form of a gummy candy that includes a biding agent, sweetener, flavoring and coloring, and a polishing agent. For example the gummy candy may include gelatin, sucrose, corn syrup, citric acid, lactic acid, natural flavors, fractionated coconut oil, and carnauba wax.

According to another implementation, the chewable composition may include a confection selected from the group consisting of: hard candy, fudge, toffee, taffy, liquorice, chocolates, marshmallows and a combination of the foregoing.

In yet another implementation, a method of forming a chewable supplement is provided. The method includes the steps of preparing a premix compound and blending a portion of the premix compound with at least one health promoting ingredient and a sweetener to form a blended slurry. After this, the blended slurry is cooked to form a cooked candy. Food acid, flavor and color are then added to the blended slurry. Next, the blended slurry is deposited onto a mold to form a shell and a flavored liquid is then deposited into the shell, where the shell and flavored liquid form a liquid-filled composition. After this step, the liquid-filled composition is cured to form a chewable supplement.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The invention may be better understood by referring to the following FIGURE. The components in the FIGURE are not necessarily to scale, emphasis instead being placed upon illustrating the principles of the invention.

FIG. 1 is a flow diagram that illustrates one example method of manufacturing a liquid-filled chewable supplement according to the principles of the present invention.

### DETAILED DESCRIPTION

The present invention relates to a liquid-filled chewable delivery system designed to enhance the delivery of dietary supplements and pharmaceutical compounds. The chewable delivery system may include a primary active ingredient (e.g., dietary supplement or pharmaceuticals) to provide the desired effect, and a delivery vehicle (i.e., the gummy candy) to contain and deliver the active ingredient to the consumer by oral ingestion.

The primary active ingredient of the present invention may include a health supplement or compound. More specifically, the active ingredient may include any combination of dietary supplements or pharmaceuticals, in liquid extract or powder form. For example, in one implementation, the active ingredient may include any combination of vitamins, nutraceuticals, minerals, antioxidants, soluble and insoluble fiber, herbs, plants, amino acids, prebiotics, probiotics, fatty acids, digestive enzymes, nutraceuticals, or any other health promoting ingredient. In another implementation, the active ingredient may include OTCs to treat symptoms of common illnesses, such as Aspirin, Benadryl®, Sudafed®, Claritin®, Maalox®, Mylanta®, Insulin, Tums®, Pepcid® AC, Monistat®, Ex-Lax®, Imodium® A.D., Robitussin®, Chloraseptic®, Thera-flu®, Alka-Seltzer, Motrin®, Dramamine®, and the like. In yet another implementation, the active ingredients may include prescription drugs such as Lipitor®, Singulair®, Lexapro, Plavix®, Morphine, Hydrocodone (Vicodin®), Demerol®, Codeine, Diazepam (Valium®), Penicillin, Prevacid®, Allegra-D®, Celebrex®, Crestor®, Valtrex®, Ambien CR®, Viagra®, Flomax®, Prozac®, and the like. Any combination of dietary supplements with pharmaceutical compounds will be dependent in part on their compatibility with the pharmaceutical compound.

As used herein, a “pharmaceutical compound” or “drug” shall include, but is not limited to, any drug, hormone, peptide, nucleotide, protein, antibody, or other chemical or biological substances used in the treatment or prevention of disease or illness, or substances which affect the structure or function of the body.

As used herein, an “over-the-counter drug” or “OTC” refers to a pharmaceutical compound, drug, or medication that may be sold directly to a consumer with a prescription from a healthcare professional.

As used herein, a “prescription drug” refers to a pharmaceutical compound, drug, or medication that may be sold only to consumers possessing a valid prescription from a healthcare professional.

As for the dosage, the active ingredients of the present invention are generally expressed in terms of grams or milligrams, but may also be expressed in active units, or international units (IU). By way of example only, a single piece of gummy candy may have 50-100 mg of Aspirin. In some implementations, the dosages of health supplements and/or pharmaceutical compounds in each gummy candy may be relatively low, allowing the consumer to adjust his/her intake of health supplements based on nutritional guidelines applicable to the particular individual.

The primary active ingredient may be delivered in a delivery vehicle that is palatable and easy to swallow. In one implementation, the delivery vehicle is chewy or gummy-like to facilitate swallowing. The delivery vehicle may include a sweetener(s), a stabilizer(s) or binder(s), a humectant(s), and/or natural and/or artificial flavors. The delivery vehicle may include natural and/or artificial colors and preservatives. In one implementation, the delivery vehicle may include glucose syrup, natural cane juice, gelatin, citric acid, lactic acid, natural colors, natural flavors, fractionated coconut oil, and carnauba wax.

### Manufacturing of Delivery System

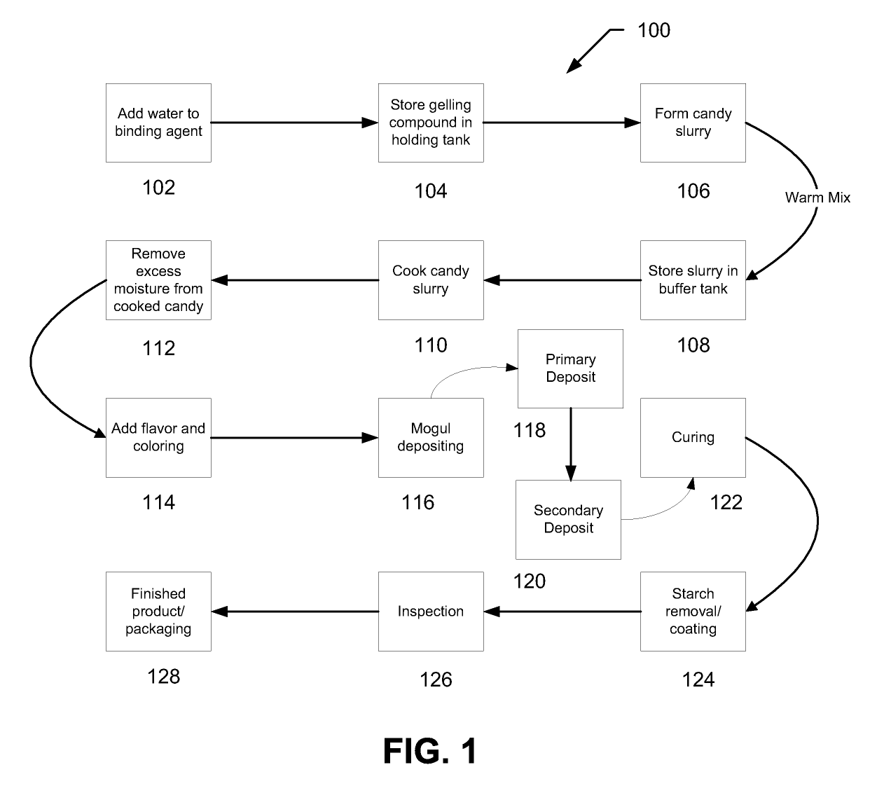

Turning now to FIG. 1, an example of a method 100 for manufacturing a gummy delivery system of the present invention is disclosed. In general, the method of manufacturing involves three main phases: (i) pre-mixing (i.e., compounding) and storing; (ii) batching and cooking; and (iii) depositing and curing.

In the first phase of pre-mixing and storing, the first step 102 includes preparing a premix compound. The premix compound may be prepared by combining water with a binding agent or gelling compound (e.g., gelatin, pectin, starch, carrageenan and/or gum) in a mixing tank, for example. The mixing tank may be any one of a plurality of different sizes. In some implementations the mixing tank may include a 1,000 gallon stainless steel planetary mixer, a scrape surface mixer, a holding tank with an agitator, or any other food-grade mixing apparatus. Although not required, in some implementations, the gelling compound may be mixed with warm water (e.g., water at an initial temperature of about 180° F.) in the mixing tank to facilitate hydration of the gelling compound; i.e., to facilitate efficient mixing of the water and the gelling compound.

During production, water and the gelling compound may be continuously mixed. For example, an agitator may be included in the mixing tank to keep the gelling compound from settling on the bottom of the tank. In some implementations, approximately 6,000 lbs to 8,000 lbs of premix compound may be produced in a period of about eight hours. In general, the gelling compound will be mixed with the water until a substantially homogeneous premix compound is formed; i.e., until the premix compound has a substantially uniform composition throughout the mixture.

As stated above, the gelling compound or binding agent may include gelatin, pectin, food starch, carrageenan, gum, or any other suitable binder, or combination thereof. For example, the binding agent may include gelatin products produced from animal sources such as beef or pork, or any other suitable gelatin product. Such products may include GELITA® Gelatine products sold by Gelita USA, Inc.

Examples of gelling compounds including pectin products may include high (methyl) ester or low (methyl) ester pectin products made from fruit sources, such as apples, apricots, carrots, citrus fruits, or any other suitable pectin product. Such products may include, for example, UNIPECTIN® HM-pectin and/or UNIPECTIN® LM-pectin products.

Examples of gelling compounds including starch ingredients may include corn starch, rice starch, potato starch, starch derivatives, and the like.

Examples of gelling compounds including carrageenan ingredients may include kappa (κ) carrageenans sold under the Gelcarin® brand, or lambda (λ) carrageenans sold under the Viscarin® brand, both available from FCM Corporation.

Depending on the binding agent used, the premix compound may include, as a non-limiting example, any one of the following formulations illustrated in Table A:

TABLE A
GELLING COMPOUND FORMULA

| | | Water |
|---|---|---|
| Binding Agent | Binding Agent (% by weight) | (% by weight) |
| gelatin | 50% | 50% |
| pectin | 2%-3% | 97%-98% |
| starch | 7%-10% | 90%-93% |
| pectin/starch | 8%-10% (1%-2% pectin/7%-8% starch) | 90%-92% |
| gelatin/starch | 7%-9% (1%-2% gelatin/6%-7% starch) | 91%-93% |
| carrageenan | 2%-5% | 95%-98% |

In some implementations, a buffer may be added to the mixing tank during preparation of the premix compound in order to regulate the pH of the premix compound. A food grade acid may be used as the buffer, such as citric acid, lactic acid, fumaric acid and/or malic acid. Other buffers include solutions of hydroxides, carbonates, citrates, phosphates, and mixtures thereof and salts thereof, e.g., sodium bisulfate and sodium citrate. As a non-limiting example, the premix compound may include approximately 0.01 to 0.03% by weight of buffer solution, or any other suitable amount for maintaining the pH of the premix compound within a range of from about 3.2 to about 4.0 during mixing.

Once the premix compound is prepared, it may then be filtered through a basket strainer (e.g., a 0.034 inch stainless steel basket strainer) or fine mesh filter material and stored in a holding tank (step 104). The holding tank may be various sizes. In one implementation, the holding tank may be a 1,500 gallon stainless steel tank. In some implementations, the holding tank may include a moderate agitator (e.g., mixing blades) for keeping the gelling compound in the premix compound from settling out of the mixture and to the bottom of the holding tank.

In the second phase of batching and cooking, at step 106 of FIG. 1, a predetermined amount of the premix compound may be delivered from the holding tank to a mixing vessel where the premix compound may be mixed and blended with various substances, including sweeteners and the primary active ingredient, i.e., nutritional supplements and/or pharmaceuticals, to form a slurry. The manner in which nutritional supplements and/or pharmaceuticals are incorporated into the gummy delivery system may depend on the heat sensitivity of the particular active ingredient. As will be discussed in greater detail below, certain ingredients that are heat resistant may be added in solid form to the mixing vessel at step 106. As a non-limiting example, 125 lbs to 185 lbs of premix compound may be delivered to the mixing vessel every 5 to 10 minutes during step 106. In some implementations, the mixing vessel in step 106 may be similar or identical in configuration to the mixing tank described above in conjunction with step 102.

In the mixing vessel, water, sweeteners, heat resistant prebiotics and/or probiotics, and additional supplements, if any, may be added to the premix compound to form a slurry mixture, for example. In one implementation, a corn syrup mix along with solid prebiotic may be added to the premix compound in step 106 and may be dissolved in the premix compound to form a slurry mixture. In one implementation, the corn syrup mix may include bulk sugar (that has been filtered and irradiated), water, corn starch, sodium citrate, corn syrup, and white grape puree. In implementations in which the active ingredient is added at step 106, the amount of active ingredient added to the premix may vary depending upon the type of chewable composition (e.g., organic or non-organic) and the desired dosage to be delivered to the consumer in the resulting chewable supplement.

Various sugars may be used as sweeteners for the gummy candy and may be added to the premix compound at step 106. Examples of appropriate sweeteners include, but are not limited to: sucrose (derived from beets or sugar cane, for example); fructose; corn syrup (which may help prevent other sugars from crystallizing in the gummy candy and may help add body to the candy, maintain moisture levels in the candy, and lower the cost of producing the candy); sorbitol, xylitol and maltitol (which are humectants); and/or various combinations of the foregoing. In one implementation, the slurry mixture may contain approximately 70% to 85% sweetener by weight, while the remaining approximately 15% to 30% of the slurry (by weight) may contain the premix compound and additives.

Prior to production, the sweeteners may be stored in bulk tanks. In one implementation, the sweetener may be stored in a holding tank at a temperature of approximately 75° F. For example, in a sweetener holding tank including corn syrup, the syrup may be irradiated by ultraviolet light to remove any contaminants in the syrup. The syrup may include high fructose corn syrup (e.g., HFCS-42, HFCS-55, or HFCS-62), glucose syrup, rice syrup, tapioca syrup, or any other suitable liquid sweetener or combination thereof. During production, the syrup may be administered to the mixing vessel manually or by automation.

Similarly, prior to production, sugar in granular form may be stored in a holding tank. During production, sugar may be fed through an automated feed system that filters the sugar to remove sediments, weighs the sugar, and delivers a desired quantity of sugar to the mixing vessel. In other implementations, sugar may be added to the mixing vessel manually.

In some implementations, various dietary supplements may by added to the premix compound at step 106, such as vitamins, minerals, fibers, herbs, plants, amino acids, antioxidants, prebiotics, probiotics, fatty acids, nutraceuticals, enzymes or any other supplements digested to promote the health and well-being of a person. Such supplements may include, but not be limited to, any of the following:

Vitamin B1 (Thiamine)
Vitamin B2 (Riboflavin)
Vitamin B3 (Niacinamide)
Vitamin B5 (Pantothenic Acid)
Vitamin B6 (Pyridoxine HCL)
Vitamin B12
Biotin
Folic Acid
Vitamin C (Ascorbic Acid/Activated C)
Calcium
Carotine
Chromium
Copper
Vitamin D (Cholecalciferol)
Vitamin E
Ginseng
Iron
Vitamin K (Phytonadione)
St. John's Wort

The above list of dietary supplements is not exhaustive, but is provided for illustrative purposes only. The length of a list of all available dietary supplements that may be utilized in the chewable composition of the invention is too lengthy to provide.

Once the premix compound is blended with the predetermined amounts of sweetener (and in some implementations, heat-resistant active ingredients), the resulting slurry may be heated to evaporate excess water, as shown in step 110 of FIG. 1. In some implementations, step 110 may include a series of substeps. In one implementation, the slurry from the mixing vessel may be processed through a magnetic device, such as a finger magnet or any other suitable magnetic device, which removes particulates in the slurry. As the slurry is processed through the magnetic device, the slurry may pass through a series of heat exchangers in order to heat the slurry to a predetermined temperature; e.g., 150° F. to 185° F. Since step 110 may include heating the slurry to relatively high temperatures, only active ingredients with a high resistance to heat (e.g., active ingredients that may withstand temperatures in excess of 200° F. without breakdown of their molecular structure) should be added at step 106 (e.g., in solid form). As the slurry passes through the series of heat exchangers, the slurry may be received by a storage buffer tank (at step 108), such as a 5,000 gallon stainless steel industrial holding tank, for example. In some implementations, the storage buffer tank may include a moderate agitator to keep any active ingredients from settling to the bottom of the storage buffer tank, for example.

From the storage buffer tank, at step 108, the warm slurry may flow to a static cooker, at step 110, where water may be evaporated from the slurry. In some implementations, evaporated water may be condensed, filtered and recycled for processing at step 102, for example. In the static cooker, in some implementations, the slurry may be cooked to a temperature of approximately 220° F. to 260° F. for approximately 30 sec. to 60 sec., until the slurry is gelatinized (i.e., dehydrated). In one implementation, the static cooker may be a 2,500 gallon high pressure steam jacketed kettle, a vacuum pressure cooker, or any other suitable cooker. In the static cooker, moisture is evaporated out of the candy slurry as the slurry is boiled. After about a minute of boiling, the slurry may consist of about a 65 to 75 brix solution.

As used herein, the term “brix” refers to the dissolved sugar-to-water ratio of a liquid or gel. For example, as described above, after boiling, in some implementations, the slurry mixture may include a ratio of dissolved sugar-to water of about 65:35 to about 75:25, on a weight/weight basis.

After the candy slurry is cooked, the cooked candy may be subjected to a vacuum, at step 112. In one implementation, the static cooker may include a vacuum apparatus. In another implementation, the cooked candy may be delivered to an industrial vacuum chamber or any other suitable enclosure including a vacuum apparatus. In the vacuum, moisture is drawn from the cooked candy by suction pressure. In some implementations, a vacuum of approximately 40 psi to 50 psi may be applied to the candy stock for approximately 15 sec. to 30 sec. However, the pressure of the vacuum and the vacuum rate will vary according to the capabilities and size of the vacuum apparatus. At this juncture, in some implementations, the cooked candy may have a brix of approximately 67 to 80, and a pH of approximately 2.8 to 4.0, for example. The cooked candy may then be filtered through a strainer.

Once cooked and filtered, as shown in step 114, the cooked candy may be transferred to a food acid tank and mixed with food acid to help control the pH of the cooked candy. Examples of food acids include: citric acid, lactic acid, fumaric acid, malic acid, ascorbic acid and the like. After adding the food acid(s), at step 114 moderately heat sensitive ingredients may be added to the cooked candy, such as various flavorings and color additives, as well as moderately heat sensitive ingredients. For example, probiotics, prebiotics, or heat sensitive drugs may also be added to the cooked candy in solid form at step 114. To help protect moderately heat sensitive active ingredients, for example drugs, the active ingredients may be encapsulated. Encapsulated active ingredients may be added at step 114 in some implementations. In some implementations, encapsulated active ingredients may be added before or during step 114. Encapsulation involves formulating a soft gel cap to cover the active ingredient, where the soft gel cap has heat resistant properties. In some implementations, the soft gel cap is a one-piece, hermetically sealed soft gelatin shell containing a liquid or semisolid called a fill. The soft gel shell may include a film-forming material such as gelatin, and a water-dispersible or water-soluble plasticizer (to impart flexibility). The soft gel shell may also include minor additives such as coloring agents, flavors, sweeteners and preservatives.

In some implementations, at step 114 the cooked candy may be passed through a trough-like apparatus known as a dosier. In the dosier, water, flavoring, coloring, and food grade acid may be added to the cooked candy to enhance the candy's taste and appearance. For example, flavoring such as artificial flavoring (i.e., mixtures of aromatic chemicals, including, but not limited to methyl anthranilate and ethyl caproate) and/or natural flavoring (i.e., flavoring obtained from fruits, berries, honey, molasses, maple sugar and the like) may be added to the cooked candy to give the candy a desired flavor. To balance the flavor (in addition to regulating the pH of the cooked candy), food grade acid may be added to the cooked candy. Such food acids may include citric acid, malic acid, lactic acid, adipic acid, fumaric acid, tartaric acid, or any other suitable food grade acid, or combination thereof. In one implementation, the flavoring, coloring, and acid may be continuously added to (e.g., dripped on) the cooked candy as the candy moves through the dosier to a starch depositor. Color additives in various combinations may be added to the cooked candy to achieve the desired color, including: red dye #40; yellow dye #5; yellow dye #6; blue dye #1, and combinations thereof. Color additives may also include natural coloring such as black carrot, annatto, tumeric, and purple berry concentrate.

The amount of flavoring, coloring, and acid added to the cooked candy at step 114 may vary according to the volume of cooked candy passing through the dosier, for example, and the desired candy formulation. As but one example, approximately 1% to 2% flavoring by weight and approximately 0.01% to 0.03% acid by weight may be added to the cooked candy composition. However, the amount of acid and flavoring added to the cooked candy formulation must be balanced to ensure the desired taste. Thus, depending on the formulation, more flavoring and less acid may be added to the cooked candy for bitter formulations, for example. For instance, to mask the flavor of a particular active ingredient in the cooked candy, a flavoring agent such as strawberry flavor or cherry flavor may be added to the mixture. The additional flavor may be adjusted based upon the active ingredient's dosage. In some instances, only food acid may be added to the cooked candy.

In some implementations, titanium dioxide may be added to the cooked candy at either step 114 to provide sheen. Those of skill in the art will recognize that various shine-enhancing agents may be utilized in conjunction with the present invention. Titanium dioxide may also stabilize the cooked candy formulation so the coloring does not bleed when it is handled, packaged, or stored.

Prior to the depositing and curing phase, the cooked candy may be subjected to quality control; i.e., the cooked candy may be checked for proper brix, pH, temperature, and proper organoleptic effects, among other characteristics.

After steps 114, the candy is ready for the depositing and curing phase, and may be transferred to a starch depositor or molding machine at step 116. In one implementation, the starch molding machine may include any commercially available starch depositing equipment (simply referred to as a “Mogul”). Thus, as shown at step 116, the cooked candy may be deposited onto a starch-coated mold to allow the cooked candy to become firm and to take on the shape of the mold.

A Mogul is a starch molding machine that automatically performs the multiple tasks involved in making gummy candy. Gummy candy may be produced in the Mogul batch-wise or via a continuous process. To start the process, the cooked candy, or gummy stock, is deposited by depositors (e.g., filling nozzles) onto starch lined trays (“mogul boards”). The mogul boards allow the cooked candy to firm and take on the shape of the tray mold, to produce a series of shaped gummy candies. In one implementation, the depositors are timed to automatically deliver the exact amount of candy needed to fill the trays as the mogul boards are passed under the depositors. In some implementations, the coloring, flavoring, and acids added to the cooked gummy candy at step 114 may be added to the candy in the depositor.

A Mogul is called a starch depositor because starch is a main component of the machine In this machine, starch has three primary purposes. First, it prevents the gummy candy stock from sticking to the mogul boards, which allows for easy removal and handling. Second, starch holds the gummy candy in place during the drying, cooling, and setting processes. Finally, starch absorbs moisture from the candies, giving them the proper texture.

In some cases, the starch used to coat the mogul boards may include recycled starch; i.e., wet starch that falls away from the candies when they are removed from the mogul boards. The re-used starch may be recycled to a starch dryer where the starch is sifted and dried. After the starch is dried, it may then be cooled in a starch cooler. The cooled starch may be sifted a second time and returned to the Mogul where it may be re-circulated once again, through the same process. The recycled starch may then be sprayed evenly on the mogul board, where the cooked candy may then be deposited onto mogul boards coated with the recycled starch.

In some implementations of the present invention, at step 116, a liquid-fill may be added to the cooked candy to form a center-fill composition or slurry. The liquid-fill may include, but not be limited to, fruit juice, vegetable juice, fruit puree, fruit pulp, vegetable pulp, vegetable puree, fruit sauce, vegetable sauce, honey, maple syrup, molasses, corn syrup, sugar syrup, polyol syrup, hydrogenated starch hydrolysates syrup, emulsions, vegetable oil, glycerin, propylene glycol, ethanol, liqueurs, sorbitol or any other liquid sweetener, dairy-based liquids such as milk or cream, or any combination thereof.

In some implementations, the center-fill slurry may be incorporated into the center of the cooked candy through a dual-step depositing process. For example, during the first depositing step, at step 118 of FIG. 1, an outer mold or candy shell consisting of the cooked candy may be deposited onto the mogul boards. Following the first depositing step, the candy may be processed through a second depositing step, at step 120, where the center-fill slurry may be deposited into the center of the shell.

In some implementations, to ensure that the center-fill slurry is suspended in the center of the candy shell (i.e., does not rise or sink into the shell), the temperature and specific gravity of the center-fill slurry may be less than that of the cooked candy. For example, in one implementation, the temperature of the center-fill deposited at step 120 may be, for example, 15° F.-30° F. less than the temperature of the deposited cooked candy, and the specific gravity of the center-fill slurry may be, for example, 5%-15% less than the center of gravity of cooked candy.

In some implementation, the center-fill slurry may comprise, for example, about 10% to 30% of the total weight of chewable composition, depending on the differences in the specific gravities of the cooked candy shell and the center fill-slurry. By way of example only, if the total weight of the chewable composition is 5 g, the weight of the cooked candy shell portion may be 3 g and the weight of the center fill may be 2 g.

In other implementations, the center-fill slurry may be incorporated into the center of the cooked candy by a single dual deposit process. In this instance, the center-fill may be incorporated into the cooked candy by a dual nozzle depositor that simultaneously deposits the cooked candy and the center-fill. Here, the active ingredients may be added to the center-fill, the cooked candy, or both. According to this implementation, the specific gravity of the center-fill may be less than the specific gravity of the cooked candy.

In some implementations, active ingredient may be blended with the liquid fill to form the center-fill slurry. In such implementations, the center-fill slurry may consist of about 25% by weight to about 40% by weight active ingredient, depending on the type (i.e., dietary supplement or pharmaceutical) and form of active ingredient used (i.e., liquid or powder). The active ingredient may include a pharmaceutical and/or any combination of vitamins, minerals, antioxidants, soluble and insoluble fiber, herbs, plants, amino acids, and digestive enzymes, or any other health promoting ingredient. In implementations where the active ingredients are heat sensitive, the active ingredients may be encapsulated within the center-fill slurry. The amount of active ingredient added to center-fill slurry will vary depending upon the type of chewable composition (e.g., organic or non-organic) and the desired dosage to be delivered to the consumer in the resulting chewable supplement. In some implementations the active ingredients may be encapsulated to manage or delay the release of the active ingredient into the mouth or throat of the consumer.

After the cooked candy is deposited onto the mogul boards, the mogul boards may be stacked, then removed from the stack (one-by-one) by a conveyor belt, and finally placed in a temperature and humidity controlled curing room, where the candy sits and cools (i.e., is cured), for approximately 24 hours to 48 hours in some implementations (step 122). However, the curing time for the cooked candy may vary based on the particular binding agent used in the candy and the temperature and humidity of the curing room. Proper curing time is necessary to solidify, or set the gummy product to ensure ease of packaging without breakage and proper yield. In some implementations, the candy may be cured in a curing room with approximately 15% to 25% humidity.

After curing, the gummy candies, firmed and having proper texture, may be moved to a section of the Mogul called the starch buck. In the starch buck, the mogul boards are inverted and the gummy candies are dumped into a tumbler machine at step 124. In one implementation, the tumbler may include a 2,000 gallon rotating drum or, in other implementations, a vibrating metal sieve. In the tumbler, the gummies may be tumbled together to remove any excess starch that adheres to the gummy candies. In some implementations, the vibrating metal sieve may include oscillating brushes for removing excess starch adhered to the gummies. In some implementations, excess starch may be removed by fast-rotating compressed air jets. Once the starch is removed, the gummies may become sticky, so the gummies may be coated with a polishing compound or lubricating agent to prevent the cooked candies from sticking together. Depending on the desired finished product or preferences, the gummies may be polished with fractionated coconut oil, linseed oil, sunflower oil, bees wax, carnauba wax, mineral oil, partially hydrogenated soybean oil, pear concentrate, confectioner's glaze or any other suitable food grade oil or combination thereof. In other implementations, the gummies may be sanded with sugar or a sugar substitute in a drum.

In implementations in which the active ingredient (e.g., drugs, probiotic(s) and/or prebiotic(s)) are particularly sensitive to heat, the active ingredient may be incorporated into the gummy delivery system in liquid form (e.g., extract) or frozen form (e.g., frozen yogurt) in a multiple-deposit step prior to curing (step 116), or after curing during coating step (step 124). In the multiple-deposit step, the gummy stock may be deposited on the mogul boards during a first deposit step. Next, the heat sensitive active ingredient(s) may be added to a syrup (e.g., a sugar and water syrup) that is deposited on the gummy stock during a second deposit step. The gummy stock is then allowed to cure, thus having an active ingredient-containing syrup coating. In alternative implementations, heat sensitive active ingredients may be added to the solid sugar particles, thus creating a sugar coating that may be applied to the gummy candy during the step 124 of the manufacturing process 100.

After the gummies are coated, they may be placed on an inspection belt, at step 126, where the candies are inspected for food safety and proper organoleptic effects. For example, on the inspection belt the gummy candies may be passed by a detector or x-ray to insure that no particulate or other foreign material has been deposited into the candy during the depositing stage. Once the candy passes inspection, it is packaged for distribution (step 128).

During packaging and storage (step 128), the finished gummy candies may be refrigerated to maintain the shelf-life and efficacy of the active ingredients, for example. Alternatively, the gummy candies may be specially packaged, for example, in a vacuum pack injected with liquid nitrogen.

The disclosure above only describes one implementation of a method of manufacturing a delivery system of the present invention. Other methods and implementations may be used to manufacture delivery systems in accordance with the present invention. For example, the various steps described in FIG. 1 may be carried out in any suitable order, there being no explicit limitations on the order of the steps set forth above.

### EXAMPLES

The following examples describe particular formulations and concentrations thereof for preparing chewable supplements containing a center-fill slurry of the present invention. Chewable supplements of the present invention may include non-organic and/or organic compositions. As used herein, the term “organic” refers to foods wherein at least 95% of its ingredients are produced using methods that do not involve modern synthetic inputs such as synthetic pesticides and chemical fertilizers, do not contain genetically modified organisms, are not processed using irradiation, industrial solvents, or chemical food additives, or as otherwise defined in the Organic Foods Production Act (OFPA) of 1990 and regulations in Title 7, Part 205 of the Code of Federal Regulations. For example, in some implementations, the chewable supplement may include a non-organic or an organic gummy candy. While the overall process of manufacturing a non-organic gummy and an organic gummy may involve the same steps as described above, the particular formulations of organic and non-organic delivery systems will differ. For non-organic formulations, at step 102, water may be mixed with any binding agent in the mixing tank. For example, the binding agent may include pectin, gelatin, starch, gum, or any combination thereof. In another implementation, the delivery system of the present invention may include an organic gummy. To create an organic gummy, the ingredients used to form the drug must meet the requirements for organic certification. As used herein, the terms “organic compliant” or “organic certification” refer to products containing (by weight or fluid volume, excluding water and salt) not less than 70% organically produced raw or processed agricultural products. These ingredients may include, but not be limited to, organic evaporated cane juice, organic tapioca syrup, organic grape juice, citric acid, lactic acid, sodium citrate, natural color (e.g., black carrot juice concentrate, annatto, turmeric, purple berry concentrate) and natural flavor (e.g., strawberry, orange, pineapple, grape), LMOs and prebiotics.

A. Vitamin formulation

In some implementations, the delivery system of the present invention may include a liquid-filled multi-vitamin. For example, a liquid-filled chewable vitamin supplement in accordance with the present invention may include the following composition:

TABLE B
VITAMIN GUMMY FORMULA

| Ingredients | Content (by Weight) |
|---|---|
| Water | 5.0% |
| Lactic acid | 1.0% |
| Citric acid | 1.0% |
| Pure cane sugar | 36.0% |
| Corn syrup | 45.0% |
| Gelatin/Pectin/Starch mix | 7.0% |
| Multi-vitamin blend (300 mg) | 3.0% |
| Flavoring | 1.5% |
| Coloring | 0.5% |

In this example, about 93 lbs of warm water may be mixed with about 7 lbs of gelatin/pectin/starch compound (“binding agent”) in the mixing tank to form 100 lbs of premix compound having a homogeneous 93/7 blend of water and binding agent.

In the mixing vessel, the premix compound may be mixed with about 5 lbs of water, 36 lbs of pure cane sugar, 45 lbs of corn syrup to form the slurry, and about 2.5 lbs to 3 lbs of proprietary vitamin blend. In one implementation, the multi-vitamin blend may include approximately 2500 IU of Vitamin A, 2 mg of Vitamin B-6, 6 meg of Vitamin B-12, 60 mg of Vitamin C, 400 IU of Vitamin D, 15 mg of Magnesium, 15 mcg of Choline, 15 mg of Zinc, 20 mg of Calcium, 150 mcg of Iodine, and 15 mcg of Inositol. To stabilize the candy slurry mixture, about 0.1% sodium citrate by weight may also be added to the slurry to maintain its pH at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry.

After the candy is cooked, the cooked candy may be sent to the vacuum, where the candy may be further dehydrated. After leaving the vacuum, the cooked candy may be placed in the dosier where about 1.5% of cranberry and orange flavoring by weight and about 0.5% of black carrot juice coloring by weight may be added to the cooked candy. To balance the flavoring, citric acid and lactic acid may be added to the cooked candy.

After adding the flavoring and coloring, the cooked candy may be deposited into the Mogul machine through, for example, a dual depositing step where an outer mold containing the cooked candy may be deposited on the Mogul board in a first depositing step, and a liquid-fill containing honey may be deposited into outer mold in a second depositing step. After the center-fill slurry is deposited onto the cooked candy, the formulation is cured.

After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### B. Drug Formulation

In some implementations, the delivery system of the present invention may include an organic gummy formulation. To create an organic gummy, the ingredients used to form the drug must meet the requirements for organic certification, as prescribed in the Organic Foods Production Act (OFPA) of 1990 and regulations in Title 7, Part 205 of the Code of Federal Regulations. For example, an organic liquid-filled chewable drug in accordance with the present invention may include the following composition:

TABLE C
ORGANIC DRUG GUMMY FORMULA

| Ingredients | Content (by Weight) |
|---|---|
| Water | 9.0% |
| Lactic acid | 1.0% |
| Citric acid | 1.0% |
| Organic evaporated cane juice | 39.3% |
| Organic tapioca syrup | 45.0% |
| Pectin | 2.0% |
| Aspirin (50 mg) | 0.2% |
| Natural flavoring | 1.5% |
| Natural coloring | 1.0% |

In this example, about 98 lbs of warm water may be mixed with about 2 lbs of gelatin/pectin/starch compound (“binding agent”) in the mixing tank to form 100 lbs of premix compound having a homogeneous 93/2 blend of water and binding agent.

In the mixing vessel, the premix compound may be mixed with about 6 lbs of water, 39.3 lbs of organic evaporated cane juice, 45 lbs of organic tapioca syrup to form the candy slurry mixture, and, about 0.15 lbs to 0.2 lbs of Aspirin.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry.

After the candy is cooked, the cooked candy may be sent to the vacuum, where the candy may be further dehydrated. After leaving the vacuum, the cooked candy may be placed in the dosier where about 1.5% of cranberry and orange flavoring by weight and about 0.5% of black carrot juice coloring by weight may be added to the cooked candy. To balance the flavoring, citric acid and lactic acid may be added to the cooked candy.

After adding the flavoring and coloring, the cooked candy may be deposited into the Mogul machine in a dual-depositing step.

After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

The examples provided above are for illustrative purposes only. Formulations for chewable supplements of the present invention may vary based on the desired dosage of pharmaceutical compounds and/or dietary supplements, additives, sweeteners, and coloring added to the composition.

While the implementations above describe a dual depositing processes for incorporating the liquid-fill into the delivery vehicle, person skilled in the art would appreciate that the liquid-fill may be incorporated into the delivery vehicle by a multi-stage processes, in particular, a process involving two or more steps. Further, while implementation of the present invention describe the active ingredients as being homogenously mixed or incorporated into the cooked candy (i.e., delivery vehicle), in other implementations, active ingredient may be mixed, blended, or otherwise incorporated into the liquid-fill.

While implementations of the invention have been described with reference to a gummy delivery system, the invention is not limited to this application and may be readily used for any chewable or digestible composition. For example, implementations of the invention may also be employed in organic, vegetarian or non-vegetarian tablets, capsules, or solid candies. For purposes of the present invention, the term “vegetarian” refers to a product or composition that does not contain any animal ingredients or by-products. The present invention may also apply to other forms of candies such as jelly beans or caramel-based candies. Further, while the dimensions of the holding and mixing vessels are provided herein by way of example only, the actual dimensions of these vessels may vary based on the amount of premix compound and candy slurry produced in a given time period (e.g., per day).

The foregoing description of implementations has been presented for purposes of illustration and description. It is not exhaustive and does not limit the claimed inventions to the precise form disclosed. Modifications and variations are possible in light of the above description or may be acquired from practicing the invention. The claims and their equivalents define the scope of the invention.

What is claimed is:

1. A chewable composition comprising:
a center-fill composition comprising a flavored liquid ingredient and at least one health promoting ingredient; and
a chewable shell surrounding the center-fill composition, the chewable shell comprising a binding agent, flavoring ingredient, and a sweetener.

2. The chewable composition of claim 1 where the health promoting ingredient comprises one or more dietary supplements selected from a group consisting of vitamins, minerals, herbs, probiotics, prebiotics, amino acids, fatty acids, digestive enzymes, food supplements or any other health promoting ingredient, and combinations thereof.

3. The chewable composition of claim 1 where the health promoting ingredient is a pharmaceutical compound.

4. The chewable composition of claim 1 where the binding agent is a gelatin base.

5. The chewable composition of claim 1 where the binding agent is a pectin base.

6. The chewable composition of claim 1 where the chewable shell comprises at least one health promoting ingredient.

7. The chewable composition of claim 1 where the flavored liquid ingredient is a syrup.

8. A chewable composition comprising:
a center-fill composition comprising a flavoring ingredient; and
a chewable shell surrounding the center-fill composition, the chewable shell comprising a binding agent, flavoring ingredient, a sweetener, and at least one health promoting ingredient.

9. The chewable composition of claim 8 where the health promoting ingredient comprises one or more dietary supplements selected from a group consisting of vitamins, minerals, herbs, probiotics, prebiotics, amino acids, fatty acids, digestive enzymes, food supplements or any other health promoting ingredient, and combinations thereof.

10. The chewable composition of claim 8 where the health promoting ingredient is a pharmaceutical compound.

11. The chewable composition of claim 8 where the binding agent is a gelatin base.

12. The chewable composition of claim 8 where the binding agent is a pectin base.

13. The chewable composition of claim 8 where the center-fill composition comprises at least one health promoting ingredient.

14. The chewable composition of claim 8 where the flavoring ingredient is a syrup.

15. A chewable composition comprising:
a center-fill composition comprising a flavored liquid ingredient and at least one health promoting ingredient; and
a chewable shell surrounding the center-fill composition, the chewable shell comprising an organic binding agent, a flavoring ingredient, and an organic sweetener.

16. The chewable composition of claim 15 where the health promoting ingredient comprises one or more dietary supplements selected a group consisting of vitamins, minerals, herbs, probiotics, prebiotics, amino acids, antioxidants, fatty acids, digestive enzymes, food supplements or any other health promoting ingredient, and combinations thereof.

17. The chewable composition of claim 15 where the health promoting ingredient is a pharmaceutical compound.

18. The chewable composition of claim 15 wherein the binding agent is a pectin base.

19. The chewable composition of claim 15 wherein the binding agent is a starch base.

20. The chewable composition of claim 15 where the chewable shell comprises at least one health promoting ingredient.

21. The chewable composition of claim 15 where the flavored liquid ingredient is a syrup.

22. A chewable candy comprising:
a center-fill composition comprising a flavoring ingredient; and
a chewable shell surrounding the center-fill composition, the chewable shell comprising a binding agent, flavoring ingredient, a sweetener, and at least one health promoting ingredient.

23. The candy of claim 22 where the health promoting ingredient comprises one or more dietary supplements selected from a group consisting of vitamins, minerals, herbs, probiotics, prebiotics, amino acids, antioxidants, fatty acids, digestive enzymes, food supplements or any other health promoting ingredient, and combinations thereof.

24. The candy of claim 22 where the health promoting ingredient is a pharmaceutical compound.

25. The chewable composition of claim 22 where the binding agent is a gelatin base.

26. The chewable composition of claim 22 where the binding agent is a pectin base.

27. The chewable composition of claim 22 where the center-fill composition comprises at least one health promoting ingredient.

28. A method of forming a chewable supplement, comprising:
preparing a premix compound;
blending a portion of the premix compound with at least one health promoting ingredient and a sweetener to form a blended slurry;
cooking the blended slurry to form a cooked candy;
adding food acid, flavor and color to the blended slurry;
depositing the blended slurry onto a mold to form a shell;
depositing a flavored liquid into the shell, the shell and flavored liquid forming a liquid filled composition; and
curing the liquid filled composition to form a chewable supplement.

29. The chewable composition of claim 28 where the health promoting ingredient comprises one or more dietary supplements selected from a group consisting of pharmaceuticals, vitamins, minerals, herbs, probiotics, prebiotics, amino acids, fatty acids, digestive enzymes, food supplements or any other health promoting ingredient, and combinations thereof.

30. The chewable composition of claim 28 where the health promoting ingredient is a pharmaceutical compound.
